(19) United States
(12) Patent Application Publication
(10) Pub. No.: US 2008/0021564 A1
(43) Pub. Date: Jan. 24, 2008

(54) Humeral head resurfacing implant and methods of use thereof

(76) Inventor: Stephen B. Gunther, Healdsburg, CA (US)

Correspondence Address: CLARK & ELBING LLP, 101 FEDERAL STREET, BOSTON, MA, 02110, US

(21) Appl. No.: 11/489,994

(22) Filed: Jul. 20, 2006

(51) Int. Cl. A61F 2/40; A61B 17/90

(52) U.S. Cl. 623/19.14; 623/23.14; 606/87

(57) ABSTRACT

The invention features a monoblock (non-modular) humeral head (shoulder) resurfacing implant designed to replace a portion of the patient's humeral head. The humeral head may need replacing due to trauma, osteonecrosis, infection, arthritis, or other causes. The implant of the invention is designed to be performed either as a hemiarthroplasty or as a component of a total shoulder replacement along with a standard glenoid or inset glenoid implant.

### FIELD OF THE INVENTION

The present invention relates to the field of shoulder replacement.

### BACKGROUND OF THE INVENTION

Shoulder replacement surgery has been an excellent treatment option for patients with disabling pain related to shoulder arthritis. Humeral implants utilized in such replacement surgeries are typically made from metal, and the implants are fixed to the bone either with bone cement (polymethylmethacrylate) or by press fitting the implant to the bone using a roughened inner surface coating on the metal for bony integration. There are two types of humeral head implants in current use: those that replace the humeral head (e.g., implants that include a humeral head and stem that is inserted into a pre-drilled (reamed) humeral shaft) and those that “resurface” the humeral head without removing much bone (e.g., implants that include a humeral head cap with a short central post; these implants do not have a stem inserted down into the humeral shaft).

Unlike classical humeral replacement implants, resurfacing humeral head implants spare a significant amount of healthy bone, thus minimizing the potential for peri-operative humeral fractures and making revision surgery, if and when necessary, easier and less risky. They are easier to remove during revision procedures than classical humeral implants that have a humeral shaft intramedullary implant attached to a humeral head implant. Humeral head resurfacing implants are typically used to replace the humeral head articular surface in patients who have isolated humeral head damage and little or no damage to the articular surface of the glenoid socket.

Current designs for humeral head resurfacing implants use a semi-spherical articular dome with a small stem for rotational stability and fixation. The inner surface of the articular head is also semi-spherical and meets with a spherically machined humeral head. Because the size and shape of the humerus and humeral head of a patient can vary greatly, due to, e.g., size, gender, bone structure, and disease, matching a properly sized prosthesis to the defect can be difficult, and typically results in the presence of voids between the bone and implanted prosthesis. These voids result in limited contact between the prosthesis and the reamed bone and may limit the load transfer capability between the prosthesis and the humerus. Modular humeral head resurfacing implants have been used to overcome these limitations, but modular implants suffer from several limitations, including, e.g., increased wear and tear, which generally results in an earlier failure time, and a reduction in load bearing capabilities. Furthermore, improper selection, placement, positioning, and fixation of the modular implant components can result in unusual stress conditions causing implant loosening and a subsequent reduction in the service life of the implant. Periodic, long-term follow-up is needed to monitor the position and state of the implant components, as well as the condition of the adjoining bone, thus causing inconvenience to the patient and a reduced quality of life.

Currently used resurfacing humeral implants are also limited in that they do not allow adequate exposure to the glenoid surface during total shoulder replacement surgeries in cases where both a glenoid implant and a resurfacing humeral implant are needed. In this situation, the remaining humeral head blocks the view (surgical exposure) to the glenoid socket. Therefore, the standard of care is to utilize resurfacing humeral implants only with a hemiarthroplasty procedure in which the glenoid is spared. If there are significant arthritic changes to both surfaces (humeral head and glenoid), then a total shoulder implant is utilized, which involves cutting away the entire humeral head in order to provide exposure to the glenoid. This requires replacement of the entire humeral head with a metal humeral head implant attached to an intramedullary humeral stem after the glenoid implant is implanted.

Thus, there remains a need for an improved resurfacing humeral head implant and improved methods for performing replacement shoulder surgery.

### SUMMARY OF THE INVENTION

The invention features a non-modular humeral head resurfacing implant, devices for preparing the humeral head for accepting the humeral head resurfacing implant, and methods of implanting the humeral head resurfacing implant.

A first aspect of the invention features a non-modular humeral head resurfacing implant which replaces part of the humeral head, including a portion of the articular surface. The implant is implanted using a procedure that involves removing and replacing part of the humeral head.

The non-modular humeral resurfacing implant of the first aspect includes a body, which has an exterior articulating surface and an internal volume, an interior surface opposite the exterior articulating surface, and a stem portion extending outwardly from a central point within the interior surface. The body is substantially hemi-spherical or elliptical in shape, while the exterior articulating surface is capable of articulating with a glenoid implant or glenoid bone of a patient. The interior surface includes a solid anterior portion that has a planar support surface for supporting the implant against a humeral head of the patient. The solid anterior portion fills at least about 20% of the internal volume of the body.

In several embodiments of the first aspect of the invention, the solid anterior portion fills at least about 20% of the internal volume of the body (preferably the solid anterior portion fills at least about 30% of the internal volume, more preferably 40% of the internal volume, and most preferably 50% or more of the internal volume); the implant is sized and shaped to conform to a humeral head of a patient following resection surgery to remove the articulating surface; and the stem has a length of less than about 50 mm (e.g., a length in the range of between about 20 to 40 mm), and a width of less than about 20 mm (e.g., a width in the range of between about 5 to 15 mm). In another embodiment of the first aspect of the invention, the implant is manufactured using polyethylene, metal, ceramic, or a combination thereof.

A second aspect of the invention features a humeral head cutting jig designed for the purpose of resecting part of the humeral head.

The purpose of this design is to replace part of the humeral head with a non-modular resurfacing implant. The partial humeral head resection allows adequate exposure to the glenoid surface for glenoid replacement.

The humeral head cutting jig, which is sized and shaped to fit onto and over the superior portion of a humeral head of a patient, includes a substantially hemispherical or elliptical body that fully or partially conforms to and encircles the superior portion of the humeral head; an extender connected to the body at a connection site (the extender extends posterior to the body and sits circumferentially in rotatable connection against an inferior portion of the humeral head); a guide track that extends circumferentially between and separates the body from the extender except at the connection site (the guide track is sized to accept a cutting device for resecting the superior portion of the humeral head); a stem attached to the body; and a handle attached to the stem.

In several embodiments of the second aspect of the invention, the stem is linearly attached to the body or is attached to the body at another angle (e.g., at a 90° or greater); and the jig further includes one or more apertures for securing the jig to the humeral head (e.g., apertures that are sized and shaped to receive K-wire, pins, or screws).

A third aspect of the invention features a method for performing joint arthroplasty, in which the humeral bone of a patient is exposed and prepared to accept the humeral implant of the first aspect of the invention. The humeral bone is resected to form a substantially planar surface and a short central elongated cavity is prepared in the humeral bone to accept a short stem or peg. The implant is then implanted onto the resected humeral bone. The implant can be fixed to the resected humeral bone by cement, e.g., using polymethylmethacrylate (PMMA) or a compatible fixation material, or by press-fit.

The method of the third aspect of the invention includes the steps of exposing the humeral bone; preparing the humeral bone by removing a portion of the humeral bone, including, e.g., the humeral head and articulating surface of the humeral bone, which forms a resected humeral bone having a planar surface; preparing a centrally located elongated cavity within the resected humeral bone, such that the resected humeral bone is capable of receiving the non-modular humeral resurfacing implant of the first aspect of the invention (including all of the embodiments described therein); and implanting the implant onto the resected humeral bone.

As used herein, the term “about” means±10% of the recited value.

By “substantially hemispherical” is meant a hemispherical shape±10° and also includes hemi-oblong and elliptical shapes.

### BRIEF DESCRIPTION OF THE DRAWINGS

FIG. 1 is the side view of a typical resurfacing humeral implant of the prior art.

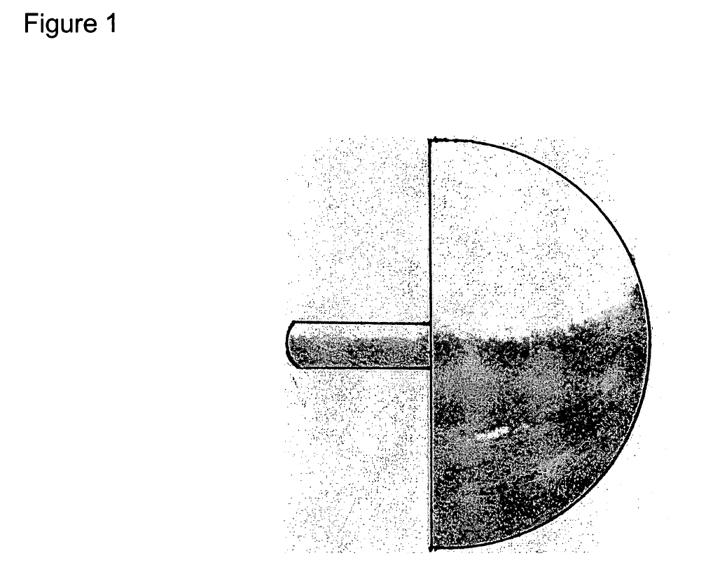

FIG. 2 is a superior view of the implant of FIG. 1.

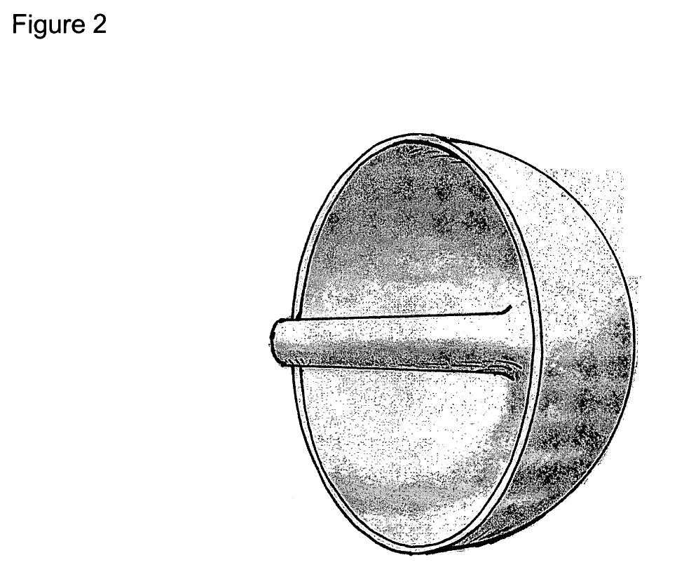

FIG. 3 is a side view partially in cross section of the monoblock (non-modular) humeral resurfacing implant of the invention.

FIG. 4 is a superior view of the monoblock (non-modular) humeral resurfacing implant of FIG. 3.

FIG. 5 is a view of the humeral cutting jig of the invention mounted on a humerus prior to resection.

FIG. 6 is a view showing resection of the humeral head by inserting a saw blade into the guide portion of the humeral cutting jig of the invention while it is mounted on the humeral head.

FIG. 7 is a view partially in cross section of the monoblock (non-modular) humeral resurfacing implant of the invention implanted on a resected humerus.

### DETAILED DESCRIPTION OF THE INVENTION

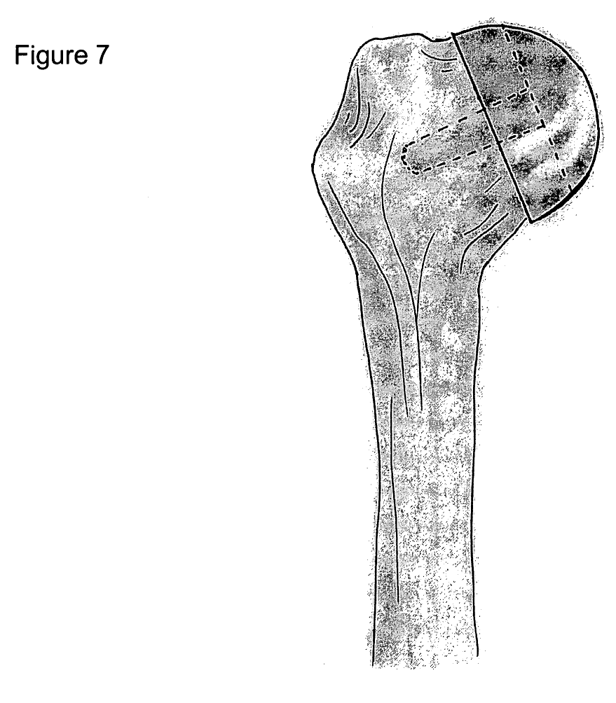

The invention features a monoblock (non-modular) humeral head resurfacing implant which replaces both the articular surface of the humeral head and a portion of the humeral head bone. The humeral head resurfacing implant of the invention is designed to be used in resurfacing surgeries where more than just the articular surface is removed, which distinguishes it from other currently used humeral resurfacing implants which only replace the articular surface. It is also unlike modular (multiple piece) humeral resurfacing implants, which are designed to replace deformed humeral bone. The humeral head resurfacing implant of the present invention is provided as a monoblock (non-modular) implant, which provides increased implant strength as compared to modular resurfacing implants. The humeral head resurfacing implant of the present invention also provides improved implant fixation strength due to its intimate association with the resected surface of the patient's humerus (see FIG. 7). The intimate association decreases the rate of implant loosening and extends the wear life of the implant.

The humeral head resurfacing implant of the invention also provides enough access to the glenoid in order to allow glenoid resurfacing when a total shoulder replacement is necessary. To fit the prosthesis to the patient's humerus, a portion of the humeral head and articulating surface is removed. Removal of only a portion of the humeral head spares the majority of the patient's bone while providing significantly improved access to the patient's glenoid. This procedure also facilitates preparation of the glenoid surface and replacement of the patient's glenoid with, e.g., a glenoid implant, such as the one disclosed in copending U.S. Ser. No. 11/066,978, which is incorporated herein by reference.

The humeral head resurfacing implant of the invention does not require the removal of the whole humeral head. The implant can be fixed using a bone cement, such as polymethylmethacrylate (PMMA) or a compatible fixation material. Alternatively, the humeral head resurfacing implant can be press-fit.

The humeral head resurfacing implant can be used in hemiarthroplasty surgeries as well as in total shoulder arthroplasty surgeries. Patients who can benefit from the use of the humeral head resurfacing implant of the invention and the improved methods using the implant include young, middle, and older patients, especially those presenting with arthritis (typical total shoulder replacement (TSR) patients) or damage or injury to the shoulder. The humeral head resurfacing implant allows hemiarthroplasty surgery and TSR surgery for new, previously contraindicated applications, including applications in which the patient presents with bone defects on the humeral head.

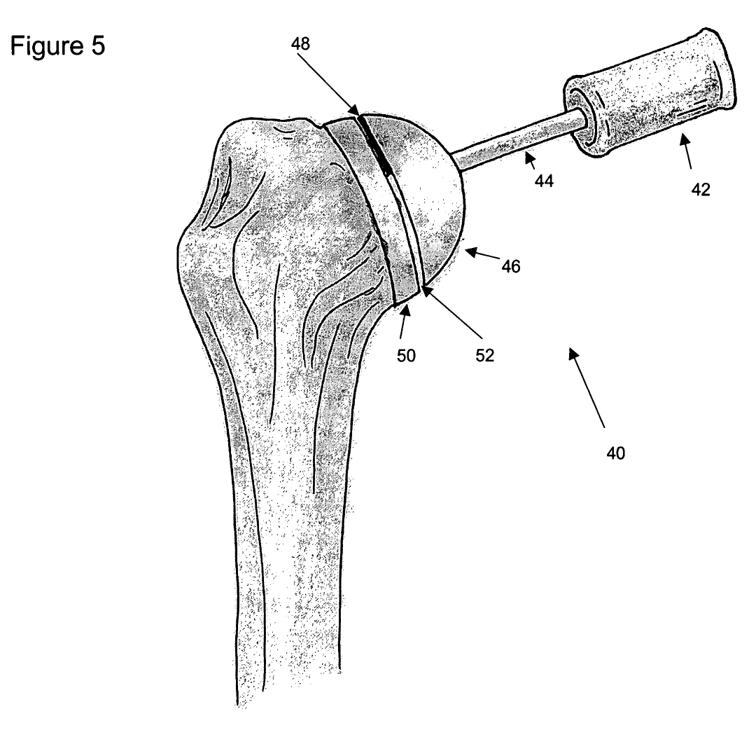

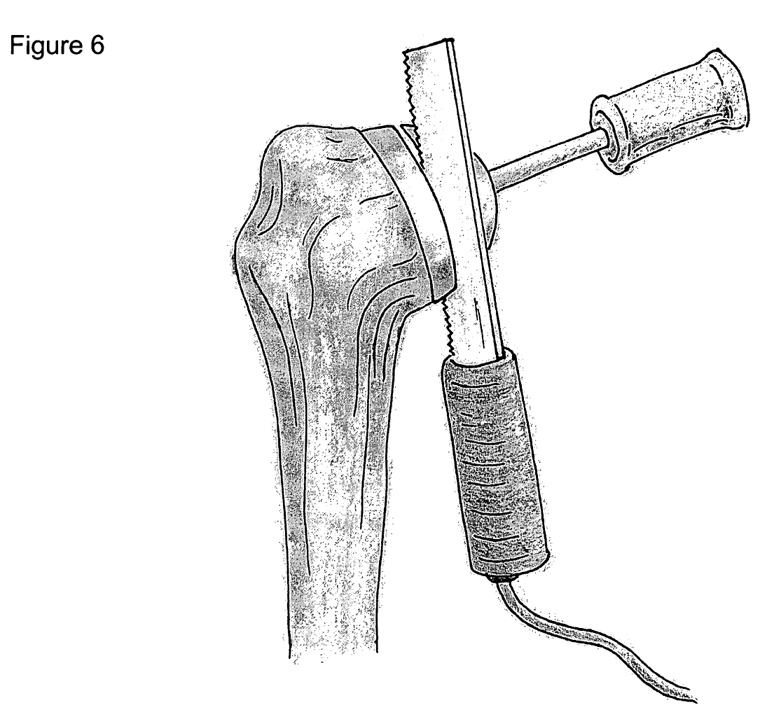

The invention also includes a humeral head cutting jig designed to resect a portion of the humeral head. The base of the jig is in rotatable connection with the humerus and includes a guide for receiving a saw blade. The jig facilitates removal of the articulating surface and a portion of the humeral head (see, e.g., FIGS. 5 and 6). Approximately 20% to 50% of the humeral head is resected during this procedure. A jig of the invention can be provided in many different sizes and shapes. A jig having the size and shape that most closely matches the humeral head of the patient can be selected for use during the resection process.

The humeral head resurfacing implant of the invention can be used in patients suffering from degenerative joint disease, such as osteoarthritis; avascular necrosis; inflammatory degenerative joint disease such as rheumatoid arthritis; and other assorted shoulder diseases and injuries. Contraindications include infection; rapid disease progression as manifested by joint destruction or bone absorption; skeletally immature patients; cases where there is inadequate neuromuscular status (e.g., prior paralysis) or poor bone stock; and neuropathic joints.

Conditions presenting increased risk of failure include marked bone loss, severe osteoporosis, or revision procedures for which an adequate fit of the prosthesis cannot be achieved; metabolic disorders that may impair bone formation; osteomalacia; and poor prognosis for good wound healing (e.g., end-stage diabetes, severe protein deficiency and/or malnutrition).

### Non-Modular Humeral Head Resurfacing Implant

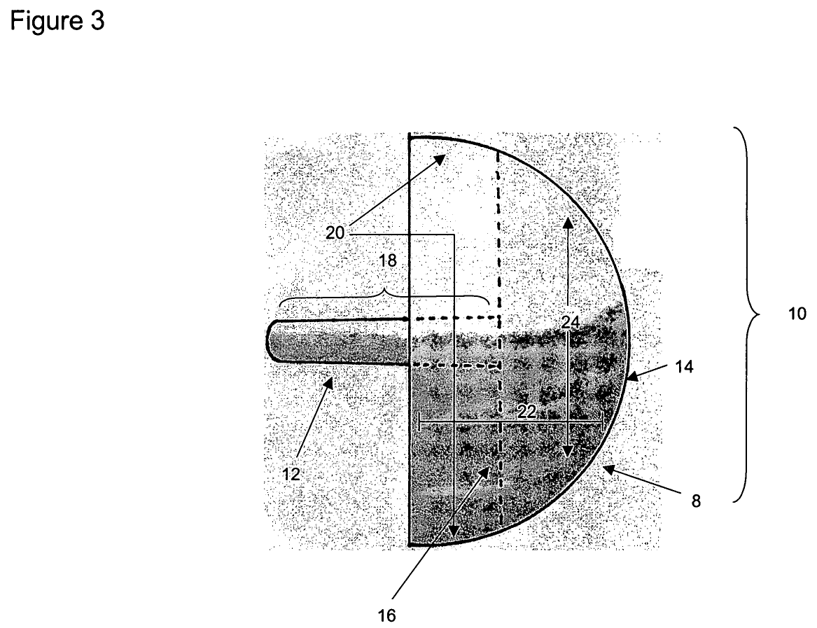

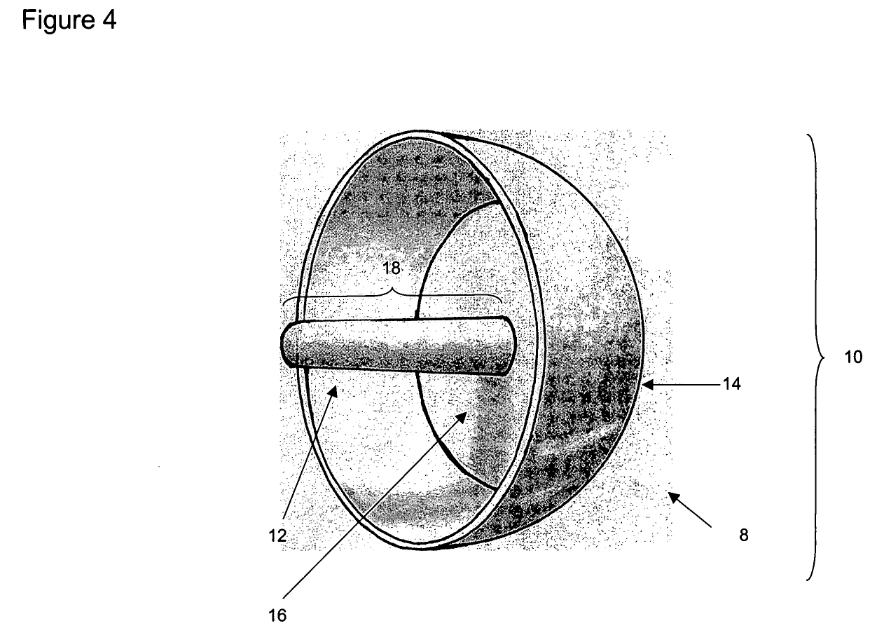

Referring now to FIGS. 3 and 4, monoblock (non-modular) humeral head resurfacing implant (10) according to the invention includes body (8), exterior articulating surface (14), which is capable of cooperating with the glenoid cavity of a patient, interior volume (22), anterior solid portion (24), interior planar surface (16), which aids in the fixation of the implant (10) to the resected humeral head of a patient, and interior sides (20). The glenoid cavity may include a glenoid prosthesis (not shown) or a natural glenoid cavity. Exterior articulating surface (14) may be hemi-spherical, substantially hemispherical, elliptical, substantially elliptical, or may have any suitable configuration capable of conforming to, and articulating with, the glenoid cavity of the patient. Interior planar surface (16) and sides (20) may be smooth or may include features, such as pores or coatings that facilitate bonding of the interior surface of implant (10) to a resected humeral head. At least about 20% of interior volume (22) of body (8) is solid (indicated as anterior solid portion (24)); anterior solid portion (24) of interior volume (22) ends at interior planar surface (16). In preferred embodiments, anterior solid portion (24) of interior volume (22) fills at least about 20% of body (8) and can fill up to about 50% or more of body (8).

Monoblock (non-modular) humeral head resurfacing implant (10) according to the invention also includes stem (12) having elongated portion (18), which is inserted at least partially into the humeral head. Stem (12) extends from interior planar surface (16). Stem (12) may optionally be tapered such that the diameter of stem (12) is at its greatest at interior planar surface (16). To facilitate cooperation between stem (12) and the implant site, stem (12) may optionally include one or more details, such as grooves, flutes, and threads. In addition, stem (12) may also include surface features, such as pores or coatings that enhance the bond between stem (12) and the implant site. The stem length is preferably less than about 50 mm (preferably between about 20-40 mm), and the stem width is preferably less than about 20 mm (preferably between about 5-15 mm).

Implant (10) can be fixed using a bone cement, such as polymethylmethacrylate (PMMA) or a compatible fixation material, or it can be press-fit. Press-fit implants of the invention are preferably coated, entirely or partially, with a porous, textured, or roughened material along the interior surface (16, 20) to improve fixation of implant (10) in the humeral head. Cement-fixed implants of the invention can have a smooth surface, a roughened, textured surface, or a mixture of both.

### Resurfacing Humeral Head Cutting Jig

Referring now to FIGS. 5 and 6, resurfacing humeral head cutting jig (40) according to the present invention, which is used during resection of the humeral head, includes body (46), which is shaped to fit onto and over the humeral head of a patient, and extender (50), which is the proximal portion of body (46) and which sits in rotatable connection against the posterior portion of the humeral head. Body (46) can be hemi-spherical, substantially hemi-spherical, elliptical, substantially elliptical, or any configuration capable of providing substantial contact with the surface of a humeral head. Extender (50) attaches to body (46) at connection site (52), but is otherwise circumferentially separated from body (46) by guide (48). Guide (48), which separates body (46) from extender (50) at all but the connection site, is a track that a cutting device can follow during resection of the humeral head using cutting jig (40) to provide a substantially planar surface on the humeral head (see FIG. 6). After the majority of the humeral cut is performed, the jig can be rotated or removed in order to finish the cut adjacent to the solid portion of the jig (i.e., the portion obscured by connection site (52)). Following resection of the humeral head using cutting jig (40), the substantially planar humeral head surface can now be fitted with the monoblock (non-modular) humeral head resurfacing implant of the invention.

Cutting jig (40) also includes handle (42) and stem (44). Handle (42) is used to secure cutting jig (40) to the humeral head of a patient during resection. Stem (44) can be linearly attached to body (46), or it can be attached to body (46) at a right (90°) or greater angle (e.g., 120°, 145°, and 160°).

Cutting jig (40) should be seated on top of the anatomic neck of the humeral head. Osteophytes which obscure the junction of the humeral head and humeral shaft should be removed to allow cutting jig (40) to sit securely on the humeral head circumferentially. Cutting jig (40) can be fixed to the humerus using wires, pins, or screws at the appropriate angle and version, if necessary. The rotator cuff should be carefully protected with retractors, and then the humeral cut is performed using a cutting device, preferably an oscillating saw, by placing the cutting device in guide (48) and following along the surface of the cutting jig. In order to finalize the cut, the jig may be rotated or removed.

The cutting jig can be manufactured using metal or any other appropriate material.

The invention will now be described by the following example. The following example is meant to illustrate the invention. It is not meant to limit the invention in any way.

### EXAMPLE

A middle aged patient presents with shoulder arthritis involving both sides of the shoulder joint (glenoid and humeral head). Because the patient is relatively young (45-65 years old), one goal of the surgery would be to conserve as much of the patient's bone as possible. However, current resurfacing humeral head implants do not allow adequate visualization (exposure) of the glenoid vault in order to implant a glenoid component, and thus, a total shoulder replacement surgery under these circumstances would involve cutting away the entire humeral head in order to provide sufficient exposure to the glenoid. This would result in a significant loss of the patient's humeral bone.

The non-modular humeral resurfacing implant of the invention would allow the surgeon to save important humeral bone because its use would require resecting only as much of the humeral head as would be required to allow glenoid visualization. Generally, this amount is about 20% to about 50% of the humeral head, rather than the entire humeral head, as is normally required when performing TSR surgery that employs currently used humeral implants. Therefore, a total shoulder implant surgery can be safely and adequately performed on a relatively young patient using the non-modular humeral resurfacing implant, jigs, and technique of the invention while minimizing bone loss and other complications.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

What is claimed is:

1. A non-modular humeral resurfacing implant comprising:
a body comprising an exterior articulating surface having a substantially hemi-spherical or elliptical shape and an internal volume, said exterior articulating surface being capable of articulating with a glenoid of a patient;
an interior surface opposite said exterior articulating surface, wherein said interior surface includes a solid anterior portion that comprises a planar support surface for supporting the implant against a humeral head of said patient, wherein said solid anterior portion fills at least 20% of the internal volume of said body; and
a stem portion extending outwardly from a central point within said interior surface.

2. The implant of claim 1, wherein said solid anterior portion fills between about 20 to about 50% of the internal volume of said body.

3. The implant of claim 1, wherein said implant is sized and shaped to conform to a humeral head of a patient following resection surgery to remove the articulating surface.

4. The implant of claim 1, wherein said stem has a length of less than about 50 mm and a width of less than about 20 mm.

5. The implant of claim 4, wherein said stem has a length in the range of between about 20 to 40 mm.

6. The implant of claim 4, wherein said stem has a width in the range of between about 5 to 15 mm.

7. The implant of claim 1, wherein said implant is manufactured using polyethylene, metal, ceramic, or a combination thereof.

8. A humeral head cutting jig sized and shaped to fit onto and over the superior portion of a humeral head of a patient, wherein said jig comprises:
a substantially hemispherical or elliptical body that fully or partially conforms to and encircles the superior portion of the humeral head;
an extender connected to said body at a connection site, wherein said extender extends posterior to the body and sits circumferentially in rotatable connection against an inferior portion of the humeral head;
a guide track that extends circumferentially between and separates the body from the extender except at the connection site, wherein the guide track is sized to accept a cutting device for resecting the superior portion of the humeral head;
a stem attached to the body; and
a handle attached to the stem.

9. The jig of claim 8, wherein said stem is linearly attached to the body.

10. The jig of claim 8, wherein said stem is attached to the body at a 90° or greater angle.

11. The jig of claim 8, wherein said jig further comprises one or more apertures for securing the jig to the humeral head.

12. The jig of claim 11, wherein said apertures are sized and shaped to receive K-wire, pins, or screws.

13. A method for performing joint arthroplasty on a humeral bone of a patient, said method comprising:
(a) exposing the humeral bone;
(b) preparing the humeral bone by removing a portion of the humeral bone comprising the humeral head and articulating surface of the humeral bone to form a resected humeral bone having a planar surface and preparing a centrally located elongated cavity within the resected humeral bone, wherein the resected humeral bone is capable of receiving a non-modular humeral resurfacing implant wherein the implant comprises:
a body comprising an exterior articulating surface having a hemi-spherical or elliptical shape and an internal volume, said exterior articulating surface being capable of articulating with a glenoid cavity of the patient;
an interior surface opposite said exterior articulating surface, wherein said interior surface includes a solid anterior portion that comprises a planar support surface for supporting the implant against the humeral bone, wherein said solid anterior portion fills at least 20% of the internal volume of said body; and
a stem portion extending outwardly from a central point within said interior surface; and
(c) implanting the implant onto the resected humeral bone.

14. The method of claim 13, wherein said solid anterior portion fills between about 20 to about 50% of the internal volume of said body.

15. The method of claim 13, wherein said implant is sized and shaped to conform to a humeral head of a patient following resection surgery to remove the articulating surface.

16. The method of claim 13, wherein said stem has a length of less than about 50 mm and a width of less than about 20 mm.

17. The method of claim 16, wherein said stem has a length in the range of between about 20 to 40 mm.

18. The method of claim 16, wherein said stem has a width in the range of between about 5 to 15 mm.

19. The method of claim 13, wherein the implant is secured to the resected humeral bone using cement or by press-fit.

20. The method of claim 13, wherein the implant is further secured using one or more surgical screws.

21. The method of claim 13, wherein the implant is manufactured using polyethylene, metal, ceramic, or a combination thereof.
